(19) United States
(12) Patent Application Publication
(10) Pub. No.: US 2021/0109120 A1
(43) Pub. Date: Apr. 15, 2021

(54) Method, Pretreatment Reagent and Kit for Detecting Drug in Hair

(71) Applicant: GUANGZHOU WONDFO BIOTECH CO.LTD., Guangdong (CN)

(72) Inventors: Qiuping ZHANG, Guangdong (CN); Yuhao HONG, Guangdong (CN); Jihua WANG, Guangdong (CN)

(21) Appl. No.: 16/894,165

(22) Filed: Jun. 5, 2020

(30) Foreign Application Priority Data
Oct. 11, 2019 (CN) 201910962991.4

(51) Int. Cl. G01N 33/94; G01N 33/53

(52) U.S. Cl. CPC G01N 33/94; G01N 33/53

(57) ABSTRACT

The present disclosure provides a sample pretreatment reagent for detecting drug in hair, which comprises a hydroxyl group-containing compound which belongs to a reducing agent capable of cleaving disulfide bonds. The disulfide bonds in hair particles are opened to expose the drug molecules embedded in hair gaps, thus improving the release amount of drug molecules from hair without affecting the structure or property of the drug molecules, so that the drug detection limit of the method for detecting drug in hair is reduced, and the drug molecules in a lower content in hair can be detected.

### TECHNICAL FIELD

The present disclosure belongs to the field of drug testing, and in particular to a method and a kit for detecting drug in hair.

### BACKGROUND

The currently available drug testing samples mainly include urine, blood, saliva and hair. The testing methods comprise chemical methods, chromatography, spectrometry, immunoassay and the like.

Most drugs are excreted through urine. Usually, only a subject's urine needs to be collected as the test sample. Using urine as a sample is low in cost and simple in operation, but with certain privacy and the possibility of fraud. Moreover, the urine sample can only reflect a drug addict's drug use in recent days and must be timely collected. Blood is also a commonly used drug testing sample which contains high concentrations of drugs and metabolites, so blood is an ideal material. However, blood can only reflect the drug abuse in recent days or even several hours, and blood contains infectious disease virus, so sampling is dangerous. Saliva which is also frequently used as a sample for drug testing is easy to collect, and the collecting device is simple and easy to purify. However, saliva as a sample is short in the testing window time, vulnerable to pollution, and low in testing sensitivity to some drugs.

Hair samples can overcome the disadvantages of many liquid samples: first, they can be stored at room temperature in dark for several years; second, drug abuse in the last several months or even in the last six months is traceable; third, the test materials are simple to take and difficult to adulterate. Drug in hair is mainly originated from the following process: drug is absorbed into blood and then diffused into hair through cell membrane, and secondly, from the following process: drug is diffused into sweat gland or sebaceous gland and then absorbed into hair by hair follicles. Drug will also be passively adsorbed on hair when there is medicine (drug) pollution outside. Hair grows at a relatively slow speed, for example, the average growth rate of hair is about 1.1 cm per month, which constitutes the theoretical basis of hair testing.

For the gold standard method of samples in hair, the LC-MS/MS testing method is currently the commonly used method recognized by the administration of justice and the labor market. The main process includes washing, drying, grinding, ultrasonic dissolution, extraction, machine operation and other steps. The operation process is extremely complex, which requires large-scale liquid chromatograph mass spectrometer, site and professionals. The results come out slowly, which is not suitable for streamlined operation.

### SUMMARY

Based on the above, the present disclosure aims to provide a simple and quick method and a kit for extracting and detecting drug in hair to be suitable for quick and streamlined drug testing in hair.

To achieve the above purpose, the present disclosure provides a specific technical scheme as follows:

a sample pretreatment reagent for detecting drug in hair, which contains a reducing agent, wherein the reducing agent is a hydroxyl group-containing compound.

The present disclosure also provides use of a hydroxyl group-containing compound as a reducing agent in the preparation of a sample pretreatment reagent for detecting drug in hair or a kit for detecting drug in hair.

The present disclosure also provides a kit for detecting drug in hair, and the specific technical scheme is as follows:

a kit for detecting drug in hair, which comprises the sample pretreatment reagent for detecting drug in hair as described above and strikers.

The present disclosure also provides a hair pretreatment method for detecting drug in hair, and the specific technical scheme is as follows:

a hair pretreatment method for detecting drug in hair, which comprises the following step: mixing a hair sample to be tested with the sample pretreatment reagent for detecting drug in hair as described above and the strikers as described above by oscillation.

The present disclosure also provides a method for detecting drug in hair, and the specific technical scheme is as follows:

a method for detecting drug in hair, which comprises the following steps:

mixing a hair sample to be tested with the sample pretreatment reagent for detecting drug in hair as described above and the strikers as described above by oscillation to obtain a solution to be tested; and

detecting the solution with an immunochromatographic strip or an immunochromatographic reagent cup or reagent card for detecting drug.

Based on the above technical schemes, the present disclosure has the following advantages:

The sample pretreatment reagent for detecting drug in hair according to the present disclosure comprises a hydroxyl group-containing compound which belongs to a reducing agent capable of cleaving disulfide bonds. The disulfide bonds in hair particles are opened to expose the drug molecules embedded in hair gaps, thus improving the release amount of drug molecules from hair without affecting the structure or property of the drug molecules, so that the drug detection limit of the method for detecting drug in hair is reduced, and the drug molecules in a lower content in hair can be detected.

The kit according to the present disclosure further comprises strike beads. The strike beads with a reasonable grain size can split hard hair tissues into turbid porridge-like liquid by mechanical action. The strike beads generate heat when striking and thus provide a suitable temperature when working with the reducing agent, which is more favorable for further hair crushing. Moreover, disulfide bonds in hair particles are opened to expose the drug molecules embedded in hair gaps, thus improving the release amount of drug molecules from hair, so that the drug detection limit of the method for detecting drug in hair is further reduced.

Moreover, by applying the pretreatment reagent for detecting drug in hair according to the present disclosure to hair pretreatment, the obtained solution to be tested is tested by immunochromatography, which is favorable for simplifying the traditional testing process with the large-scale liquid chromatograph mass spectrometer, thereby accelerating testing, allowing streamlined operation, and suitable for on-site timely large-batch screening.

### BRIEF DESCRIPTION OF THE DRAWINGS

FIG. 1 is a schematic diagram of sample treatment result of hair treatment with the glass beads as the strike beads;

FIG. 2 is a schematic diagram of sample treatment result of hair treatment with the zirconium beads as the strike beads;

FIG. 3 is a schematic diagram of sample treatment result of hair treatment with the steel beads as the strike beads;

FIG. 4 is a schematic diagram of sample treatment result of hair treatment with mixed strike beads comprising strike beads with a grain size of 2.0-2.2 mm and strike beads with a grain size of 3.0-3.2 mm in a number ratio of 20:0;

FIG. 5 is a schematic diagram of sample treatment result of hair treatment with mixed strike beads comprising strike beads with a grain size of 2.0-2.2 mm and strike beads with a grain size of 3.0-3.2 mm in a number ratio of 15:5;

FIG. 6 is a schematic diagram of sample treatment result of hair treatment with mixed strike beads comprising strike beads with a grain size of 2.0-2.2 mm to strike beads with a grain size of 3.0-3.2 mm in a number ratio of 10:10;

FIG. 7 is a schematic diagram of sample treatment result of hair treatment with mixed strike beads comprising strike beads with a grain size of 2.0-2.2 mm to strike beads with a grain size of 3.0-3.2 mm in a number ratio of 5:15;

FIG. 8 is a schematic diagram of sample treatment result of hair treatment with mixed strike beads comprising strike beads with a grain size of 2.0-2.2 mm to strike beads with a grain size of 3.0-3.2 mm in a number ratio of 0:20;

FIG. 9 is a structure diagram of the immunochromatographic reagent cup;

FIG. 10 is a structure diagram of the immunochromatographic reagent card;

FIG. 11 is the testing result of the negative reference material after maltose cracking;

FIG. 12 is the testing result of the positive reference material after maltose cracking;

FIG. 13 is the testing result of the negative reference material after maltose cracking in the presence of dispersing agent CF;

FIG. 14 is the testing result of the positive reference material after maltose cracking in the presence of dispersing agent CF.

### DETAILED DESCRIPTION

To make the present disclosure easy to understand, a more comprehensive description of the present disclosure will be given below with reference to the embodiments, and better embodiments of the present disclosure are given below. However, the present disclosure can be implemented in many different forms and shall not limited to the embodiments described herein. The purpose of providing these embodiments is to provide a more thorough and comprehensive understanding of the disclosure of the present disclosure. It should be understood that the experimental methods with specific conditions not indicated in the following embodiments are usually in accordance with the conventional conditions or the conditions recommended by the manufacturer. Various commonly used reagents used in the embodiments are commercially available products.

Unless otherwise defined, all technical and scientific terms used herein have the same meanings as those generally understood by those skilled in the art of the present disclosure. The terms used in the specification of the present disclosure are only intended to describe the purposes of specific embodiments, rather than limiting the present disclosure. The term “and/or” as used herein includes any and all combinations of one or more related listed items.

A sample pretreatment reagent for detecting drug in hair of the present disclosure contains a reducing agent which is a hydroxyl group-containing compound.

Preferably, the mass percentage of the hydroxyl group-containing compound in the sample pretreatment reagent is 0.1%-20%. The color development will be deeper if the mass percentage exceeds this range, which is easy to lead to a false-negative result.

Preferably, the hydroxyl group-containing compound is at least one selected from the group consisting of ascorbic acid or reducing sugar.

More preferably, the reducing sugar is maltose.

Preferably, the sample pretreatment reagent further contains a dispersing agent and a buffer solution, wherein the dispersing agent is an anionic dispersing agent or a non-ionic dispersing agent.

More preferably, the dispersing agent is a carboxylate substance. More preferably, the dispersing agent is the dispersing agent CF. The product used in the specific embodiments of the present disclosure is named CH7000 and was purchased from Takemoto Oil & Fat Co., Ltd.

Further preferably, the sample pretreatment reagent comprises the following components:

a reducing agent with a mass percentage of 0.1%-10%;

a dispersing agent with a mass percentage of 0.1%-10%; and

a TRIS buffer solution of 0.02M-1M.

A kit for detecting drug in hair of the present disclosure comprises the sample pretreatment reagent for detecting drug in hair as described in any of the items above and strike beads.

Optionally, the grain size of the strike beads ranges from 1.8 to 3.4 mm.

Optionally, the strike beads have at least one grain size. The strike beads may have one grain size, or two, three or four grain sizes. Preferably, when the strike beads have only one grain size, the grain size thereof ranges from 2.8 to 3.4 mm, preferably 3-3.2 mm.

Preferably, the strike beads comprise first strike beads and second strike beads, wherein the grain size of the first strike beads range from 1.8 to 2.4 mm, and the grain size of the second strike beads range from 2.8 to 3.4 mm.

More preferably, the grain size of the first strike beads ranges from 2.0 to 2.2 mm, and the grain size of the second strike beads ranges from 3.0 to 3.2 mm.

Preferably, the number ratio of the first strike beads to the second strike beads is 15:5-1:19.

More preferably, the number ratio of the first strike beads to the second strike beads is 12:8-8:12. Further preferably, the number ratio of the first strike beads to the second strike beads is 10:10.

Preferably, the concentration ratio of the strike beads to the buffer solution is that: 18-22 strike beads are contained in every 2 ml of buffer solution. More preferably, 20 strike beads are contained in every 2 ml of buffer solution.

Preferably, the strike beads are: glass beads, zirconium beads or steel beads. More preferably, the strike beads are zirconium beads.

The present disclosure also provides a hair pretreatment method for detecting drug in hair, which comprises the following steps: mixing a hair sample to be tested with the sample pretreatment reagent for detecting drug in hair as described in any of the items above by oscillation.

Preferably, the mixing by oscillation is: mixing by oscillation at a speed of 2500-3500 rpm for 100-150s.

The present disclosure also provides a kit for detecting drug in hair, which comprises the sample pretreatment reagent for detecting drug in hair as described above.

Preferably, the kit further comprises: an immunochromatographic strip or an immunochromatographic reagent cup or reagent card for detecting drug.

Preferably, the immunochromatographic test strips used for detecting drug comprises a sample pad, a glass cellulose membrane, a nitrocellulose membrane and a piece of absorbent paper which are successively arranged on a base plate; wherein the nitrocellulose membrane comprises a test area and a control area, wherein the test area is coated with drug molecule-coupled BSA, and the control area is coated with goat anti-mouse antibody; and the glass cellulose membrane contains colloidal gold particle-labeled materials, wherein the colloidal gold labeled materials comprise a colloidal gold-labeled mouse IgG antibody and a colloidal gold-labeled anti-drug molecule antibody.

In some other embodiments, as an alternative to the colloidal gold label, labeling methods such as ELISA and the like which are commonly used by other immunochromatographic test strips can be used.

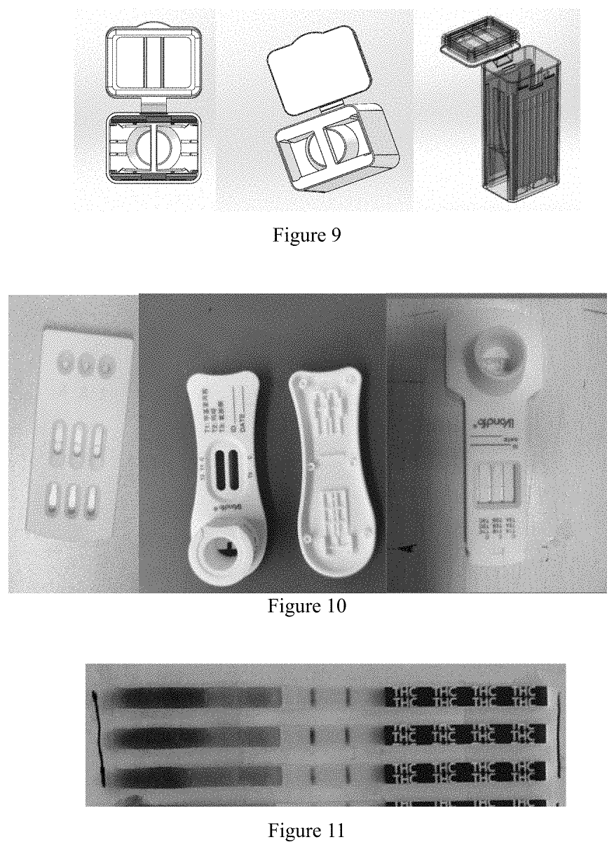

In some of the embodiments, the structure of the immunochromatographic reagent cup is as shown in FIG. 9. The type of the immunochromatographic reagent card is as shown in FIG. 10.

The present disclosure also provides a method for detecting drug in hair, which comprises the following steps: mixing a hair sample to be tested with the sample pretreatment reagent for detecting drug in hair as described in any of the items above by oscillation to obtain a solution to be tested; detecting the solution with an immunochromatographic strip or immunochromatographic reagent cup or reagent card for detecting drug.

### Example 1 Effect of the Material of Strike Beads in the Pretreatment Kit for Drug in Hair

This example provided a pretreatment kit for detecting drug in hair which comprised strike beads and a hair pretreatment reagent. The effects of different strike bead materials on the testing sensitivity were tested as follows:

Multiple portions of hair were added into hair treatment tubes respectively, and then strike beads with a grain size of 3.0-3.2 mm and those with a grain size of 2.0-2-2 mm were added at an addition number ratio of 10:10, with a total of 20 beads. 2 ml of hair pretreatment reagent was added into the treatment tube, and then the mixture was shaken at 3,200 rpm for 120 s with an on-site hair drug tester. The hair pretreatment reagent contained 1% of a reducing agent, 0.2% of a dispersing agent and 0.2M of a buffer solution, wherein the reducing agent was ascorbic acid, the dispersing agent was dispersing agent CF (a polycarboxylic acid derivative), and the component of the buffer solution was a TRIS buffer solution.

Detection was performed with an immunochromatographic reagent cup or reagent card. The immunochromatographic reagent cup or reagent card comprised a sample pad, a glass cellulose membrane containing colloidal gold particle-labeled materials, a nitrocellulose membrane and a piece of absorbent paper which were successively connected and arranged on a base plate. The nitrocellulose membrane comprised a test area coated with amphetamine (AMP) (a drug molecule)-coupled BSA and a control area coated with goat anti-mouse antibody. The colloidal gold particle-labeled materials comprised a colloidal gold-labeled mouse IgG antibody and a colloidal gold-labeled anti-drug molecule antibody. The strike beads were glass beads, zirconium beads and steel beads respectively, and the results are compared as shown in Table 1:

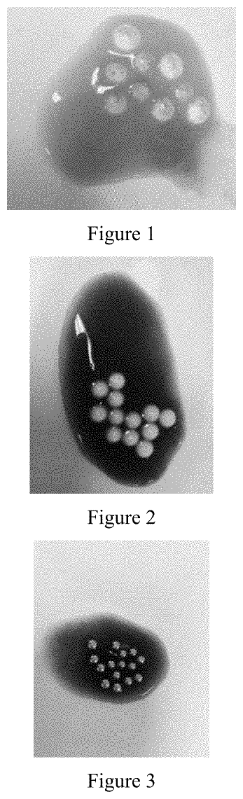

TABLE 1
Comparison of Testing Effects of Strike Beads with
Different Materials on the Same Hair Sample

| | | | | Zirconium | | | |
|---|---|---|---|---|---|---|---|
| Treatment Mode | | Glass Beads | | Beads | | Steel Beads | |
| After strike | | FIG. 1 | | FIG. 2 | | FIG. 3 | |
| | | Beads were worn | | Beads were | | Beads were intact, but the | |
| | | and broken | | intact | | tube cap was broken | |
| Performance | Positive enterprise | +1.5 | 3/3 | +1.5 | 3/3 | +1.5 | 3/3 |
| evaluation | reference material P1 | | | | | | |
| | Negative enterprise | +5 | 3/3 | +5 | 3/3 | +5 | 3/3 |
| | reference material N1 | | | | | | |

*Note:
“+5 3/3” indicates that the color development results in 3 repetitions are all +5. +5 indicates the depth of color development. The greater the number is, the deeper the color development is, and the range is “+0~+5”.

Different strike beads have no effect on the performance test, but there are differences in physical properties. Glass beads are easy to break, and steel beads have higher bearing force to the pressure from the tube wall due to high hardness. The zirconium beads are preferred under the existing conditions.

### Example 2 Effect of the Grain Size and Mixing Ratio of Strike Beads in the Kit for Detecting Drug in Hair

This example provided a pretreatment kit for detecting drug in hair which comprised strike beads and a hair pretreatment reagent. The effects of different strike bead grain sizes and mixing ratios on the testing sensitivity were tested as follows:

Multiple portions of hair were added into hair treatment tubes respectively, and then a total of 20 mixed strike beads comprising strike beads with a grain size of 2.0-2-2 mm and those with a grain size of 3.0-3.2 mm were added. The addition number ratios of the two kinds of strike beads with different grain sizes were 20:0, 15:5, 10:10, 5:15 and 0:20 respectively. 2 ml of hair pretreatment reagent was added into the treatment tube, and then the mixture was shaken at 3,200 rpm for 120s with an on-site hair drug tester. The hair pretreatment reagent contained a reducing agent, a dispersing agent and a buffer solution. The reducing agent was ascorbic acid, the dispersing agent was dispersing agent CF (a polycarboxylic acid derivative), and the component of the buffer solution was a TRIS buffer solution.

Detection was performed with an immunochromatographic reagent cup or reagent card, and the results are shown in Table 2.

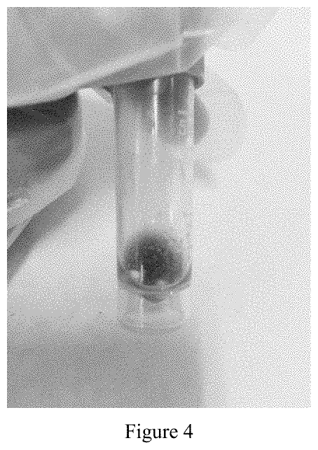

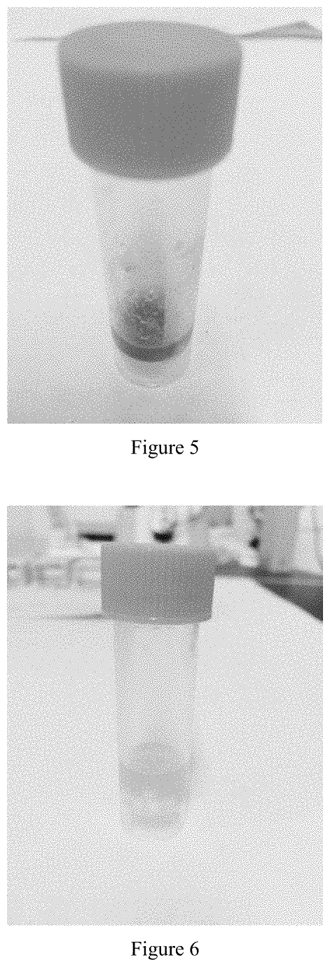

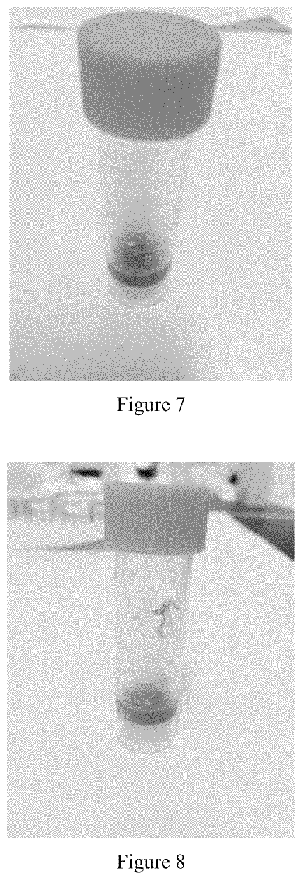

TABLE 2
Comparison of Testing Effects of Strike Beads in Different Ratios on the Same Hair Sample

| | | Treatment Mode | | | | | | | | | |
|---|---|---|---|---|---|---|---|---|---|---|---|
| | | | | 15:5 | | | | 5:15 | | 0:20 | |
| | | | | Hair was | | | | Hair was | | Hair was | |
| | | 20:0 | | cracked, but | | | | cracked, but | | cracked, but | |
| | | Hair was | | short hair was | | 10:10 | | stratification | | short hair was | |
| | | not | | visible to the | | Completely | | occurred | | visible to the | |
| | | completely | | naked eye on | | homogeneous | | when left | | naked eye on | |
| State of the hair liquid after | | cracked | | the tube wall | | liquid | | standing | | the tube wall | |
| treatment | | (FIG. 4) | | (FIG. 5) | | (FIG. 6) | | (FIG. 7) | | (FIG. 8) | |
| Performance | positive | +3 | 3/3 | +2 | 3/3 | +1 | 3/3 | +1.5 | 3/3 | +1.5 | 3/3 |
| evaluation | reference | | | | | | | | | | |
| | material | | | | | | | | | | |
| | negative | +5 | 3/3 | +5 | 3/3 | +5 | 3/3 | +5 | 3/3 | +5 | 3/3 |
| | reference | | | | | | | | | | |
| | material | | | | | | | | | | |

*Note:
“+5 3/3” indicates the number of test repetitions, that is, the color development results in 3 repetition tests are all +5. +5 indicates the depth of color development. The greater the number is, the deeper the color development is, and the range is “+0~+5”.

Result: Different ratios of the strike beads have significant effects on the testing results. The mixed strike beads of two sizes have the best effects on performance, followed by the large strike beads, and the small strike beads alone have the worst effects on testing results. So, mixed strike beads comprising an equal number of large strike beads and small strike beads are preferred.

### Example 3 Effects of the Components of the Hair Pretreatment Reagent in the Kit for Detecting Drug in Hair

This example provided a pretreatment kit for detecting drug in hair which comprised strike beads and a hair pretreatment reagent. The effects of different components of the hair pretreatment reagent on the testing sensitivity were tested as follows:

Multiple portions of hair were added into hair treatment tubes respectively, and then strike beads with a grain size of 3.0-3.2 mm and those with a grain size of 2.0-2-2 mm were added at an addition number ratio of 10:10, with a total of 20 beads. 2 ml of hair pretreatment reagent was added into the treatment tube, and then the mixture was shaken at 3,200 rpm for 120s with an on-site hair drug tester. The hair pretreatment reagent was 0.01M of HCl, 0.01M of NaOH, 1 mg/ml of keratinase and a hydroxy compound, respectively.

The operation steps for different hair pretreatment reagents are as follows, respectively:

Acid hydrolysis: 2 ml of 0.01M of HCl was added as a hair pretreatment reagent to the hair sample, and the mixture was shaken with the on-site hair drug tester for 120s. The pH was adjusted to 7.0 with 0.1M of NaOH, and then detection was performed with the immunochromatographic reagent cup.

Alkaline hydrolysis: 2 ml of 0.01M of NaOH was added as a hair pretreatment reagent to the hair sample, and the mixture was shaken with the on-site hair drug tester for 120s. The pH was adjusted to 7.0 with 0.1M of HCl, and then detection was performed with the immunochromatographic reagent cup.

Enzyme hydrolysis: 2 ml of 1 mg/ml of keratinase was added as a hair pretreatment reagent to the hair sample, and the mixture was shaken with the on-site hair drug tester for 120s. The pH was adjusted to 7.0 with 0.1M of NaOH, and then detection was performed with the immunochromatographic reagent cup.

Cracking with the hydroxyl compound: 2 ml of 1% of ascorbic acid was added as a hair pretreatment reagent to the hair sample, and the mixture was shaken with the on-site hair drug tester for 120s, and then detection was performed with the immunochromatographic reagent cup.

The results are shown in Table 3:

TABLE 3
Comparison of Detection Effects of Different Hair Pretreatment
Reagents in the Pretreatment Kit on the Same Hair Sample

| | | Treatment Mode | | | | |
|---|---|---|---|---|---|---|
| | | \textcircled{1} | \textcircled{2} | \textcircled{3} | \textcircled{4} | \textcircled{5} |
| | | Acid | Alkaline | Enzyme | Maltose | Maltose cracking + |
| | | hydrolysis | hydrolysis | hydrolysis | cracking | dispersing agent CF |
| State of the hair liquid | | Completely | Completely | Completely | Completely | Completely |
| after treatment | | homogeneous | homogeneous | homogeneous | homogeneous | homogeneous |
| | | liquid | liquid | liquid | liquid | liquid |
| Performance | positive | +1 3/3 | +1 3/3 | +2 3/3 | +0 3/3 | +0 3/3 |
| evaluation | reference | | | | | |
| | material | | | | | |
| | negative | +5 3/3 | +5 3/3 | +5 3/3 | +5 3/3 | +5 3/3 |
| | reference | | | | | |
| | material | | | | | |

| | | | | | |
|---|---|---|---|---|---|
| Physical index | With hair | With hair | With hair | With hair | Without hair |
| | residues | residues | residues | residues | residues, bright |
| | | | | | white background |

*Note:
“+5 3/3” indicates the number of test repetitions, that is, the color development results in 3 repetition tests are all +5.
+5 indicates the depth of color development. The greater the number is, the deeper the color development is, and the range is “+0~+5”.

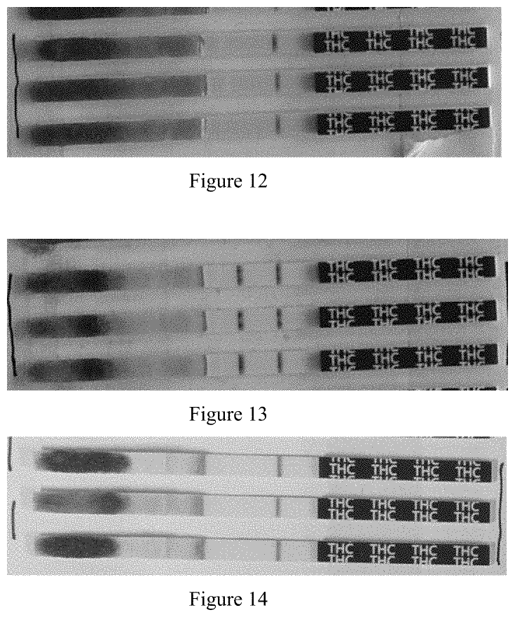

Result: Different pretreatments have significant effects on the testing results. Acid or alkaline hydrolysis is feasible. However, the concentration of drug detected in the positive sample was lower than that in the same sample cracked by the hydroxy compound (maltose). The acid or alkaline is not as effective as the hydroxy compound at extracting drug from hair. The effect of enzyme hydrolysis is the worst because the enzyme reaction takes more time. Therefore, cracking with a hydroxyl compound is preferred. In addition, the color developed in the background where a dispersing agent substance is added is more bright white. The results are shown in FIG. 11-14, wherein FIG. 11 is the testing result of the negative reference material after maltose cracking, FIG. 12 is the testing result of the positive reference material after maltose cracking, FIG. 13 is the result of the negative reference material after maltose cracking in the presence of dispersing agent CF, and FIG. 14 is the result of the positive reference material after maltose cracking in the presence of dispersing agent CF.

### Example 4 Blank Hair Sample Test

Multiple portions of blank hair (non-drug abusers' hair, without any drug such as morphine, crystal meth and ketamine) were added into hair treatment tubes respectively. Strike beads with a grain size of 3.0-3.2 mm and those with a grain size of 2.0-2-2 mm were added at an addition number ratio of 10:10, with a total of 20 beads. 2 ml of pure water was added as a hair pretreatment reagent into the treatment tube, and then the mixture was shaken at 3,200 rpm for 120 s with an on-site hair drug tester.

The test design scheme is shown in Table 4 below:

| S.N. | Setting Mode |
|---|---|
| 1# | Water |
| 2# | Water + blank hair |
| 3# | Water + CUTOFF standard |
| 4# | Water + blank hair + CUTOFF standard* |
| 5# | Water + blank hair + 1.2X CUTOFF standard |

*Note: Drug standards are certified reference materials (CRMs) calibrated by LC-MS/MS originated from cerilliant. *CUTOFF standard refers to the standard with a detection limit concentration. In this example, detection was performed for drug AMP, and the standard refers to the standard AMP with a detection limit concentration.

Testing:

Detection was performed with an immunochromatographic reagent cup or reagent card, and the result was read in 5 min. The immunochromatographic reagent cup or reagent card comprised a sample pad, a glass cellulose membrane containing colloidal gold particle-labeled materials, a nitrocellulose membrane and a piece of absorbent paper which were successively connected and arranged on a base plate. The nitrocellulose membrane comprised a test area coated with AMP (a drug)-coupled BSA and a control area coated with goat anti-mouse antibody. The colloidal gold particle-labeled materials comprised a colloidal gold-labeled mouse IgG antibody and a colloidal gold-labeled anti-AMP (a drug) antibody.

The test results are shown in Table 5:

| | | Color Development |
|---|---|---|
| S.N. | Setting Mode | Depth |
| 1# | Water | +5 |
| 2# | Water + blank hair | +5 |
| 3# | Water + CUTOFF standard | +0 |
| 4# | Water + blank hair + | +1 |
| | CUTOFF standard | |
| 5# | Water + blank hair + 1.2X | +0 |
| | CUTOFF standard | |

It can be seen that the addition of blank hair has no effects on negative samples. However, the positive sample to which blank hair was added became darker in color than that of the control sample. It was not until the concentration of the standard increased to 1.2 times that the color development became +0, which indicates that the presence of hair in the sample solution to be tested has effects on color development during testing.

After centrifugation of water+blank hair+CUTOFF standard of 4 #, the supernatant was taken as 6 #, and the testing effect is shown in Table 6 below:

| | | Color Development |
|---|---|---|
| S.N. | Setting Mode | Depth |
| 4# | Water + blank hair + CUTOFF standard | +1 |
| 6# | (Water + blank hair + CUTOFF standard) | +4 |
| | supernatant | |

It can be seen from the above results that the color development of the supernatant 6 # is three gradients deeper than that of the solution 4 #. It is suspected that the drug molecules stick to the hair, which results in less standards in the sample pad that are bound to the colloidal gold antibody by chromatography when immunochromatography is used for testing, thus making the color development darker during testing.

According to the structural properties of hair and the characteristics of protein, a hydroxyl compound (water+ascorbic acid) was added into the hair pretreatment reagent, and the testing results are shown in Table 7 below:

| | | Color Development |
|---|---|---|
| S.N. | Setting Mode | Depth |
| 4# | Water + blank hair + standard | +1 |
| 5# | (Water + blank hair + | +4 |
| | standard)supernatant | |
| 7# | Water + ascorbic acid | +5 |
| 8# | Water + blank hair + ascorbic acid | +5 |
| 9# | Water + blank hair + | +0 |
| | ascorbic acid + standard | |

Ascorbic acid has no effect on the reagent cup itself and the negative sample testing. The great advantage is that it can make the color lighter and improve the sensitivity of the product without affecting the negative color development.

### Example 5 Preparation Method of a Strip for Detecting Drug in Hair

Preparation of a Sample Pad

The components of a treatment solution for a sample pad of a strip for detecting drug in hair are shown in Table 8 below:

TABLE 8

| Substance | Content |
|---|---|
| Tris | 10 mM |
| HPMC | 0.05% |
| PAA | 1% |
| Rabbit serum | 5% |
| S9 | 1% |
| Na₂CO₃ | 0.1M |
| pH | 8.4 |

All the above raw materials were common reagents commercially available.

The above reagents were mixed, and the pH was adjusted to 8.4. The mixture was then uniformly coated on a glass cellulose membrane (100 g/m²) according to the above table at a coating concentration of 30 ml/cm². Dried in the air at 25° C. for 18-22h for standby use.

Preparation of a Gold Label Pad

A colloidal gold-labeled mouse IgG antibody probe was generated by labeling mouse IgG with colloid gold particle protein (the minimum dosage was 12 μg/ml) according to conventional gold particle labeling methods in the range of pH7.0-7.5. The above gold particles were coated on a glass cellulose membrane (100 g/m²) respectively at a coating concentration of 30 ml/cm². Dried in the air at 25° C. for 18-22h at a humidity of 10%-30% for standby use.

Coating of a Nitrocellulose Membrane

Drug molecule antigen-BSA was diluted with a coating buffer solution (the main components were TRIS buffer solution and sucrose). The dilution was sprayed onto a nitrocellulose membrane carefully and evenly at a membrane-liquid amount of 0.18 ul/mm. The membrane was placed at 25° C. with a humidity of 10%˜30%, and dried for 18-22 h.

The sample pad, gold label pad, nitrocellulose membrane and absorbent paper were pasted on a base plate in sequence, and then a desiccant was added to obtain a test strip according to the present disclosure.

Preparation of a Hair Pretreatment Reagent

The buffer solution and the hydroxyl compound were mixed using pure water in a certain ratio to form a hair pretreatment reagent. The hair pretreatment reagent and hair were mixed in a ratio of 1:10 (ml:mg), and the mixture was put into an on-site hair drug tester for treatment.

### Example 6 Testing of the Effect of the Strip for Detecting Drug in Hair

50 healthy persons were selected as control and 50 different positive samples were selected for clinical trials. The negative samples were originated from healthy people who did not take addictive drugs or other chemical drug in the company; and the clinical positive samples were originated from clinical samples collected by the domestic drug rehabilitation center or the United States and had been subjected to LC-MS/MS hair qualitative determination by a third-party testing organization in accordance with SF/Z JD0107004-2016. The treated clinical sample was poured into a testing reagent cup. The sample moved along the test strip to the glass cellulose membrane and the nitrocellulose membrane due to the capillary action. The results began to be displayed when the sample completely passed through the glass cellulose membrane and the nitrocellulose membrane. The display results were observed in 5 minutes (Note: the color development is invalid in 10 minutes). The test results of clinical negative samples are shown in Table 9, and the test results of clinical positive samples are shown in Table 10.

TABLE 9

| | | Example | Comparative Example |
|---|---|---|---|
| | | Test Strip | Test Strip |
| Sample | Sample Type | (With maltose) | (Without maltose) |
| N1 | Brown | +5 | +5 |
| N2 | Brown | +4 | +4 |
| N3 | Brown | +5 | +5 |
| N4 | Brown | +5 | +5 |
| N5 | Brown | +5 | +5 |
| N6 | Brown | +4 | +4 |
| N7 | Yellow | +4 | +4 |
| N8 | Yellow | +4 | +4 |
| N9 | Yellow | +4 | +4 |
| N10 | Yellow | +4 | +4 |
| N11 | Yellow | +4 | +4 |
| N12 | Yellow | +5 | +5 |
| N13 | Rose gold | +5 | +5 |
| N14 | Black-white | +5 | +5 |
| | hair | | |
| N15 | Black-white | +5 | +5 |
| | hair | | |
| N16 | Black-white | +5 | +5 |
| | hair | | |
| N17 | Silver grey | +5 | +5 |
| N18 | Silver grey | +5 | +5 |
| N19 | Black hair | +5 | +5 |
| N20 | Black hair | +5 | +5 |
| N21 | Black hair | +5 | +5 |
| N22 | Black hair | +4 | +4 |
| N23 | Black hair | +4 | +4 |
| N24 | Red hair | +4 | +4 |
| N25 | Black hair | +4 | +4 |
| N26 | Black hair | +4 | +4 |
| N27 | Black hair | +4 | +4 |
| N28 | Black hair | +4 | +4 |
| N29 | Black hair | +5 | +5 |
| N30 | Black hair | +5 | +5 |
| N31 | Black hair | +5 | +5 |
| N32 | Black hair | +5 | +5 |
| N33 | Black hair | +5 | +5 |
| N34 | Black hair | +4 | +4 |
| N35 | Black hair | +4 | +4 |
| N36 | Black hair | +4 | +4 |
| N37 | Black hair | +4 | +4 |
| N38 | Black hair | +4 | +4 |
| N39 | Black hair | +4 | +4 |
| N40 | Black hair | +4 | +4 |
| N41 | Black hair | +4 | +4 |
| N42 | Black hair | +4 | +4 |
| N43 | Black hair | +4 | +4 |
| N44 | Black hair | +5 | +5 |
| N45 | Black hair | +5 | +5 |
| N46 | Black hair | +5 | +5 |
| N47 | Black hair | +5 | +5 |
| N48 | Black hair | +5 | +5 |
| N49 | Black hair | +5 | +5 |
| N50 | Black hair | +5 | +5 |

Note:
N represents the sample number of a healthy person.

It can be seen from the above table that regardless of the presence or absence of a hydroxyl compound in the hair pretreatment reagent, the color development of the testing results of negative samples is basically the same.

TABLE 10

| | | Test | Test | | | |
|---|---|---|---|---|---|---|
| | | Strip | Strip | | LC-MS/MS | |
| | Sample | (With | (Without | LC-MS/MS | Threshold | |
| Sample | Type | Maltose) | Maltose) | (ng/mg) | Standard | |
| W18-01 | MOP | +0 | +0 | Detected | 0.5 | ng/mg |
| W18-02 | MOP | +0 | +1 | Detected | | |
| W18-03 | MOP | +0 | +0 | Detected | | |
| W18-04 | MOP | +0 | +0 | Detected | | |
| W18-05 | MOP | +0 | +0 | Detected | | |
| W18-06 | MOP | +0 | +0 | Detected | | |
| W18-07 | COC | +0 | +0 | Detected | 0.5 | ng/mg |
| W18-08 | COC | +0 | +1 | Detected | | |
| W18-09 | COC | +0 | +0 | Detected | | |
| W18-10 | COC | +0 | +1 | Detected | | |
| W18-11 | COC | +0 | +0 | Detected | | |
| W18-12 | COC | +0 | +0 | Detected | | |
| W18-13 | COC | +0 | +0 | Detected | | |
| W18-14 | COC | +0 | +0 | Detected | | |
| W18-15 | COC | +0 | +1 | Detected | | |
| W18-16 | COC | +0 | +0 | Detected | | |
| W18-17 | COC | +0 | +0 | Detected | | |
| W18-18 | COC | +0 | +0 | Detected | | |
| W18-19 | COC | +0 | +0 | Detected | | |
| W18-20 | COC | +0 | +0 | Detected | | |
| W18-21 | AMP | +0 | +0 | Detected | 0.5 | ng/mg |
| W18-22 | AMP | +0 | +0 | Detected | | |
| W18-23 | AMP | +0 | +0 | Detected | | |
| W18-24 | AMP | +0 | +0 | Detected | | |
| W18-25 | AMP | +0 | +0 | Detected | | |
| W18-26 | AMP | +0 | +1 | Detected | | |
| W18-27 | AMP | +0 | +0 | Detected | | |
| W18-28 | AMP | +0 | +0 | Detected | | |
| W18-29 | AMP | +0 | +0 | Detected | | |
| W18-30 | AMP | +0 | +0 | Detected | | |
| W18-31 | AMP | +0 | +0 | Detected | | |
| W18-32 | AMP | +0 | +0 | Detected | | |
| W18-33 | THC | +1 | +1 | Detected | 0.05 | ng/mg |
| W18-34 | THC | +0 | +1 | Detected | | |
| W18-35 | THC | +0 | +0 | Detected | | |
| W18-36 | THC | +1 | +1 | Detected | | |
| W18-37 | THC | +0 | +1 | Detected | | |
| W18-38 | THC | +1 | +1 | Detected | | |
| W18-39 | THC | +0 | +0 | Detected | | |
| W18-40 | MET | +0 | +0 | Detected | 0.5 | ng/mg |
| W18-41 | MET | +0 | +0 | Detected | | |
| W18-42 | MET | +0 | +0 | Detected | | |
| W18-43 | MET | +0 | +1 | Detected | | |
| W18-44 | MET | +0 | +1 | Detected | | |
| W18-45 | MET | +0 | +0 | Detected | | |
| W18-46 | BUP | +0 | +0 | Detected | 0.04 | ng/mg |
| W18-47 | BUP | +0 | +1 | Detected | | |
| W18-48 | BUP | +0 | +0 | Detected | | |
| W18-49 | PCP | +0 | +0 | Detected | 0.3 | ng/mg |
| W18-50 | PCP | +0 | +0 | Detected | | |

It can be seen from the above table that “+0” means no color development, that is, the drug content is higher than the detectable detection limit of the test strip. “+1” means color development. When the drug content is lower than the detectable detection limit of the test strip, color develops. The positive threshold standard in the table refers to the recognized positive threshold in the industry when LC-MS/MS is used for testing, that is, if a value is greater than this value, it is positive. The results show that treatment of hair with a pretreatment solution comprising a hydroxyl compound (maltose) in the hair pretreatment reagent allows the detection of drugs in lower concentrations when the hair is detected by a test strip, leading to a high detectable rate of drug. However, treatment of hair with a pretreatment solution comprising no hydroxyl compound is such that drugs in lower concentrations are un-detectable when the hair is detected by a test strip, leading to a low detectable rate of drug and a low sensitivity.

In the above 100 samples, the positive consistency rate of the kit of the present disclosure and that of the gold standard LC-MS/MS are compared as shown in Table 11 below.

Hair pretreatment reagent without maltose:

TABLE 11

| | |
|---|---|
| Kit of the Present | LC-MS/MS* |

| Disclosure | Positive | Negative | Total |
|---|---|---|---|
| Positive | 37 | 0 | 37 |
| Negative | 13 | 50 | 63 |
| Total | 50 | 50 | 100 |
| Positive | 74% | Negative | 100% |
| consistency rate | | consistency rate | |

Hair pretreatment reagent with maltose:

TABLE 12

| | |
|---|---|
| Kit of the Present | LC-MS/MS* |

| Disclosure | Positive | Negative | Total |
|---|---|---|---|
| Positive | 47 | 0 | 47 |
| Negative | 3 | 50 | 53 |
| Total | 50 | 50 | 100 |
| Positive | 94% | Negative | 100% |
| consistency rate | | consistency rate | |

Note:
The positive column contains all samples of different items.

It can be seen that the detection results of the kit of the present disclosure and that of the gold standard LC-MS/MS have a high consistency rate, which is 20% higher than the positive consistency rate of the control group. The gold standard quantitative determination of hair is unaccepted because of the great deviation among various testing organizations. At present, all the domestic gold standard LC-MS/MS methods are qualitative hair detections. Therefore, it is impossible to conclude whether the specific content values of the 3 samples are lower than the detection limit of the reagent or higher than the detection limit of the reagent.

In general, it shows that the sensitivity of the reagent has a high consistency rate with the gold standard, meets the clinical sample determination and enables the testing with good sensitivity and accuracy. The process is simple, and no large-scale liquid chromatography-mass spectrometry (LC-MS), site and professionals are required. The results come out fast, which is suitable for streamlined operation.

The technical characteristics of the above-mentioned embodiments can be combined arbitrarily. To simplify the description, not all possible combinations of the technical characteristics of the above-mentioned embodiments are described. However, the combinations of these technical characteristics should be considered as the scope recorded in this specification provided that there is no contradiction between them.

The above-mentioned embodiments only express several preferred embodiments of the present disclosure, which are described in a more specific and detailed manner, but this cannot be understood as a restriction to the scope of the patent for disclosure. It should be pointed out that several deformations and improvements can be further made for those skilled in the art without departing from the concept of the present disclosure, and all of these belong to the protection scope of the present disclosure. Therefore, the protection scope of the patent for disclosure shall be subject to the appended claims.

What is claimed is:

1. A sample pretreatment reagent for detecting drug in hair, containing a reducing agent which is a hydroxyl group-containing compound.

2. The sample pretreatment reagent for detecting drug in hair according to claim 1, wherein the hydroxyl group-containing compound is at least one selected from the group consisting of ascorbic acid and reducing sugar.

3. The sample pretreatment reagent for detecting drug in hair according to claim 2, wherein the reducing sugar is maltose.

4. The sample pretreatment reagent for detecting drug in hair according to claim 1, wherein the mass percentage of the hydroxyl group-containing compound in the sample pretreatment reagent is 0.1%-20%.

5. The sample pretreatment reagent for detecting drug in hair according to claim 1, wherein the sample pretreatment reagent further contains a dispersing agent and a buffer solution, wherein the dispersing agent is an anionic dispersing agent or a non-ionic dispersing agent.

6. The sample pretreatment reagent for detecting drug in hair according to claim 5, wherein the sample pretreatment reagent comprises the following components:
a reducing agent with a mass percentage of 0.1%-10%;
a dispersing agent with a mass percentage of 0.1%-10%; and
a TRIS buffer solution of 0.02M-1M.

7. The sample pretreatment reagent for detecting drug in hair according to claim 6, wherein the dispersing agent is a carboxylate substance.

8. Use of a hydroxyl group-containing compound as a reducing agent in the preparation of a sample pretreatment reagent for detecting drug in hair or a kit for detecting drug in hair.

9. A kit for detecting drug in hair, comprising the sample pretreatment reagent for detecting drug in hair according to claim 1 and strikers.

10. The kit for detecting drug in hair according to claim 9, wherein the strikers are strike beads having a grain size ranging from 1.8 to 3.4 mm.

11. The kit for detecting drug in hair according to claim 10, wherein the strike beads comprise first strike beads and second strike beads, wherein the first strike beads have a grain size ranging from 1.8 to 2.4 mm, and the second strike beads have a grain size ranging from 2.8 to 3.4 mm;
the number ratio of the first strike beads to the second strike beads is 15:5-1:19.

12. The kit for detecting drug in hair according to claim 10, wherein the strike beads are: glass beads, ceramic beads, zirconium beads or steel beads.

13. The kit for detecting drug in hair according to claim 12, wherein the kit further comprises: an immunochromatographic strip or an immunochromatographic reagent cup or reagent card for detecting drug.

14. A hair pretreatment method for detecting drug in hair, comprising the following steps: mixing a hair sample to be tested with the sample pretreatment reagent for detecting drug in hair according to claim 1 and strikers by oscillation;
the strikers are strike beads having a grain size ranging from 1.8 to 3.4 mm.

15. The hair pretreatment method according to claim 14, wherein the strike beads comprise first strike beads and second strike beads, wherein the first strike beads have a grain size ranging from 1.8 to 2.4 mm, and the second strike beads have a grain size ranging from 2.8 to 3.4 mm; and the number ratio of the first strike beads to the second strike beads is 15:5-1:19.

16. The hair pretreatment method according to claim 14, wherein the mixing by oscillation is: mixing by oscillation at a speed of 2500-3500 rpm for 100-150 s.

17. A method for detecting drug in hair, comprising the following steps:
mixing a hair sample to be tested with the sample pretreatment reagent for detecting drug in hair according to claim 1 and strike beads by oscillation to obtain a solution to be tested; and
detecting the solution with an immunochromatographic strip or an immunochromatographic reagent cup or reagent card or a test card for detecting drug.

18. The method according to claim 17, wherein the strike beads have a grain size ranging from 1.8 to 3.4 mm.

19. The method according to claim 17, wherein the strike beads comprise first strike beads and second strike beads, wherein the first strike beads have a grain size ranging from 1.8 to 2.4 mm, and the second strike beads have a grain size ranging from 2.8 to 3.4 mm; and the number ratio of the first strike beads to the second strike beads is 15:5-1:19.
